(19) United States
(12) Patent Application Publication
(10) Pub. No.: US 2023/0122706 A1
(43) Pub. Date: Apr. 20, 2023

(54) PATIENT BEHAVIOR MONITORING

(71) Applicant: BLUEWIND MEDICAL LTD., Herzlia (IL)

(72) Inventors: Dan Lemaitre, Park City, UT (US); Gur Oron, Tel Aviv (IL); Roy Katz, Lincoln, MA (US)

(73) Assignee: BLUEWIND MEDICAL LTD., Herzlia (IL)

(21) Appl. No.: 17/838,655

(22) Filed: Jun. 13, 2022

Related U.S. Application Data
(60) Provisional application No. 63/255,499, filed on Oct. 14, 2021.

(51) Int. Cl. A61B 5/00; A61N 1/05; A61N 1/36

(52) U.S. Cl. CPC A61B 5/4848; A61N 1/0551; A61N 1/36007; A61N 1/36139; A61N 1/36171

(57) ABSTRACT

A method for assessing treatment of a subject who has overactive bladder (OAB) includes using an implant to stimulate a tibial nerve of the subject according to a stimulation protocol. An electronic processor is used to receive, from one or more sensors, data indicative of activity of the subject over a time period extending over at least one week. Based on the data, a characteristic of a response of the subject to the stimulation is assessed. Other embodiments are also described.

### CROSS-REFERENCES TO RELATED APPLICATIONS

The present application claims priority from U.S. Provisional Application 63/255,499, filed Oct. 14, 2021, entitled, “PATIENT BEHAVIOR MONITORING,” which is assigned to the assignee of the present application and is incorporated herein by reference.

### FIELD OF THE INVENTION

Some applications of the present invention relate in general to monitoring patients who have overactive bladder (OAB).

### BACKGROUND

Overactive bladder (OAB) is characterized by increased frequency of urination, to such a degree that it negatively affects a patient’s quality of life. Four main symptoms of OAB are: (i) urinary frequency of more than eight times per day, (ii) increased urgency of urination, (iii) nocturia that adversely affects the patient’s quality of life, and (iv) involuntary release of urine that is accompanied by a feeling of urgency to urinate (urge incontinence).

Treatments for OAB include pharmacologic treatment, neuromodulation and lifestyle interventions such as dietary restrictions (e.g., fluid limitation, elimination of dietary caffeine), timed voiding and exercise to strengthen the pelvic floor muscle.

U.S. Pat. 9,713,707 to Oron et al. describes an electrostimulator implant that comprises (i) an implant body, the implant being injectable into tissue of a subject along a longitudinal axis of the implant body, (ii) first and second electrodes, disposed on respective first and second portions of the implant body; (iii) circuitry, disposed inside the implant body, and configured to drive the electrodes to apply current to the tissue; and (iv) a mesh, disposed over at least 50 percent of the implant body, and configured to serve as an anchor of the implant. Other embodiments are also described.

### SUMMARY OF THE INVENTION

Applications of the present invention are directed to apparatus and methods for assessing a subject’s response to treatment (e.g., tibial nerve stimulation) for overactive bladder (OAB).

An electronic processor receives data (e.g., GPS-derived data), that indicate the subject’s activity, from one or more sensors. Since the subject’s activities of daily living may correlate with the subject’s response to OAB treatment, the electronic processor uses the data to assess the subject’s response to the treatment. For some applications, at least some of the received data do not directly indicate a frequency of the subject’s specific voiding episodes.

For some applications, a treatment protocol (e.g., a tibial nerve stimulation protocol) is modified in response to the assessment, and the patient’s response to the modified protocol is then assessed. An indication of the assessment may be outputted to the subject and/or to a caregiver.

There is therefore provided, in accordance with an application of the present invention, a method for assessing treatment of a subject who has overactive bladder (OAB), the method comprising:
using an implant, stimulating a tibial nerve of the subject according to a stimulation protocol; and
using an electronic processor:
   receiving data from one or more sensors, the data being indicative of activity of the subject over a time period extending over at least one week, and
   based on the data, assessing a characteristic of a response of the subject to the stimulation.

In an application, receiving the data comprises receiving data not indicative of a frequency of specific voiding episodes of the subject.

In an application, receiving the data comprises receiving data indicative of a frequency of specific voiding episodes of the subject.

In an application, receiving the data comprises receiving data indicative of a cumulative duration of time during which the subject is not at home.

In an application, receiving the data comprises receiving data indicative of a cumulative duration of time during which the subject is not in a workplace of the subject.

In an application, receiving the data comprises receiving data indicative of a cumulative duration of time during which the subject is not indoors.

In an application, receiving the data comprises receiving data indicative of a cumulative duration of a plurality of periods of motion of the subject.

In an application, receiving the data comprises receiving data indicative of a frequency of nocturnal wakefulness episodes of the subject.

In an application, the method further comprises receiving the data comprises receiving data indicative of a cumulative duration of a plurality of nocturnal wakefulness episodes of the subject.

In an application, the method further comprises outputting to the subject an indication of the assessment.

In an application, the method further comprises, responsively to the assessment, outputting to the subject a modification of the stimulation protocol.

In an application, the method further comprises, responsively to the assessment, outputting to the subject a recommendation to consult with a caregiver.

In an application, receiving the data comprises receiving data indicative of a level of physical activity of the subject.

In an application, receiving the data comprises receiving data indicative of a number of steps of the subject.

In an application, receiving the data comprises receiving accelerometer-derived data.

In an application, receiving the data comprises receiving gyroscope-derived data.

In an application, receiving the data comprises receiving smartphone-derived data.

In an application, receiving the data comprises receiving microphone-derived data.

In an application, receiving the data comprises receiving the data from a personal electronic device.

In an application, receiving the data comprises receiving the data from a wearable electronic device.

In an application, receiving the data comprises receiving the data from a smartphone.

In an application, receiving the data comprises receiving the data from a watch.

In an application, receiving the data comprises receiving the data from the one or more sensors:
prior to a first time that the tibial nerve is stimulated using the implant, and
subsequently to the first time that the tibial nerve is stimulated using the implant.

In an application, the method further comprises implanting the implant adjacent to the tibial nerve, and receiving the data comprises receiving the data from the one or more sensors:
prior to the step of implanting, and
subsequently to the step of implanting.

In an application:
receiving the data from the one or more sensors comprises receiving data indicative of a distance traveled by the subject, and
assessing the characteristic of the response of the subject comprises comparing (a) data indicative of a post-treatment-initiation distance traveled by the subject subsequently to the first time that the tibial nerve is stimulated using the implant to (b) data indicative of a baseline distance traveled by the subject prior to the first time that the tibial nerve is stimulated using the implant.

In an application:
the step of receiving comprises receiving data indicative of a post-treatment-initiation distance traveled by the subject by vehicular transport, and
the step of assessing comprises identifying a positive result of the stimulation based at least in part on the post-treatment-initiation distance being greater than the baseline distance traveled by the subject by vehicular transport during the time period.

In an application:
the step of receiving comprises receiving data indicative of a post-treatment-initiation distance traveled by the subject by foot, and
the step of assessing comprises identifying a positive result of the stimulation based at least in part on the post-treatment-initiation distance being greater than the baseline distance traveled by the subject by foot during the time period.

In an application, receiving data from the one or more sensors comprises receiving data indicative of a distance traveled by the subject.

In an application:

the step of receiving comprises receiving data indicative of a distance traveled by the subject by vehicular transport.

In an application:

the step of receiving comprises receiving data indicative of a distance traveled by the subject by foot.

In an application, receiving the data indicative of the distance traveled comprises receiving GPS-derived data.

In an application, assessing the characteristic of the response of the subject to the stimulation based on the data comprises identifying a positive result of the stimulation based at least in part on a post-treatment-initiation distance traveled by the subject subsequently to the first time that the tibial nerve is stimulated using the implant being greater than a predetermined threshold distance during a given time period.

In an application:
the step of receiving comprises receiving data indicative of a post-treatment-initiation distance traveled by the subject by vehicular transport, and
the step of assessing comprises identifying a positive result of the stimulation based at least in part on the post-treatment-initiation distance being greater than the threshold distance traveled by the subject by vehicular transport during the time period.

In an application, the threshold distance is at least 50 km.

In an application, the step of identifying comprises identifying a positive result of the stimulation based at least in part on the post-treatment-initiation distance being at least 50 percent greater than the threshold distance traveled by the subject by vehicular transport during the time period.

In an application:
the step of receiving comprises receiving data indicative of a post-treatment-initiation distance traveled by the subject by foot, and
the step of assessing comprises identifying a positive result of the stimulation based at least in part on the post-treatment-initiation distance being greater than the threshold distance traveled by the subject by foot during the time period.

In an application, the step of identifying comprises identifying a positive result of the stimulation based at least in part on the post-treatment-initiation distance being at least 50 percent greater than the threshold distance traveled by the subject by foot during the time period.

In an application, the threshold distance is at least 1 km.

In an application, the method further comprises, based on the data, calculating a quality-of-life score indicative of a level of recovery of the subject from OAB.

In an application, the method further comprises, responsively to the quality-of-life score, outputting to the subject a modification of the stimulation protocol.

In an application, the method further comprises, responsively to the quality-of-life score, outputting to the subject a recommendation to consult with a caregiver.

In an application, the method further comprises outputting to the subject an indication of the quality-of-life score.

In an application:
the time period is a first time period,
the stimulation protocol is a first stimulation protocol,
the step of assessing comprises assessing the characteristic of the response of the subject to the stimulation over the first time period, and the method comprises:
   responsively to the step of assessing, determining a second stimulation protocol,
   using the implant, stimulating the tibial nerve according to the second stimulation protocol over a second time period extending over at least one week,
   using the electronic processor, receiving a second set of data from the one or more sensors, the data being indicative of activity of the subject over the second time period, and
   based on the second set of data, assessing the characteristic of the response of the subject to the stimulation over the second time period.

In an application:
stimulating the tibial nerve according to the first stimulation protocol comprises stimulating the tibial nerve during a first number of daily stimulation sessions, and
the step of stimulating the tibial nerve according to the second stimulation protocol comprises stimulating the tibial nerve during a second number of daily stimulation sessions.

In an application, the method further comprises:
determining a parameter representing a difference between:
   the characteristic of the response of the subject to the stimulation over the first time period, and
   the characteristic of the response of the subject to the stimulation over the second time period; and
outputting to the subject an indication of the parameter.

In an application, determining the second stimulation protocol comprises determining a change in a parameter of internal operation of the implant.

In an application:
stimulating the tibial nerve according to the first stimulation protocol comprises stimulating the tibial nerve at a first amplitude of individual current pulses,
determining the change in the parameter comprises determining a change in the amplitude of individual current pulses, and
the step of stimulating the tibial nerve according to the second stimulation protocol comprises stimulating the tibial nerve at a second amplitude of individual current pulses.

In an application:
stimulating the tibial nerve according to the first stimulation protocol comprises stimulating the tibial nerve at a first pulse frequency of applied current,
determining the change in the parameter comprises determining a change in the pulse frequency of applied current, and
the step of stimulating the tibial nerve according to the second stimulation protocol comprises stimulating the tibial nerve at a second pulse frequency of applied current.

There is further provided, in accordance with an application of the present invention, apparatus for use with a subject who has overactive bladder (OAB), the apparatus comprising:
an implant, the implant comprising:
   one or more electrodes,
   at least one implant antenna configured to receive wireless power, and
   implant circuitry configured to use the received wireless power to drive the one or more electrodes to apply a current to a tibial nerve of the subject;
a portable controller, comprising:
   one or more controller antennas, at least one of the controller antennas configured to transmit the wireless power,
   a battery configured to store battery power, and
   controller circuitry configured to use the battery power to wirelessly power the implant by driving at least one of the controller antennas to transmit the wireless power according to a stimulation protocol; and
an electronic processor that is configured to:
   receive data from one or more sensors, each of the sensors being configured to collect data indicative of activity of the subject over a time period extending over at least one week, and
   based on the data, assess a characteristic of a response of the subject to the stimulation.

In an application, the one or more sensors comprise a GPS signal receiver.

In an application, the one or more sensors comprise a gyroscope.

In an application, the one or more sensors comprise an accelerometer.

In an application, the one or more sensors comprise a sleep monitor.

In an application, the one or more sensors comprise a toilet-use monitor.

In an application, the one or more sensors do not comprise a toilet-use monitor.

In an application, the one or more sensors comprise a microphone.

In an application, the electronic processor is configured to modify the stimulation protocol, responsively to the assessment.

In an application, the electronic processor is configured to:
receive the data from the one or more sensors regarding:
   a first time period that is prior to a first time that the tibial nerve is stimulated using the implant, and
   a second time period that is subsequent to the first time that the tibial nerve is stimulated using the implant; and
assess the characteristic of the response of the subject to the stimulation by comparing data regarding the first time period to data regarding the second time period.

In an application, the apparatus further comprises a personal electronic device that comprises the one or more sensors.

In an application, the apparatus further comprises a wearable electronic device that comprises the one or more sensors.

In an application, the apparatus further comprises a smartphone that comprises the one or more sensors.

In an application, the apparatus further comprises a watch that comprises the one or more sensors.

In an application, the apparatus further comprises a relay unit configured to relay the data from the one or more sensors to the electronic processor.

In an application, the apparatus further comprises a housing in which the electronic processor and the relay unit are disposed.

In an application, the relay unit comprises a user interface that is configured to output to the subject an indication of the assessment.

In an application, the apparatus further comprises a housing, and the portable controller and the electronic processor are disposed within the housing.

In an application, the portable controller comprises a user interface that is configured to output to the subject an indication of the assessment.

The present invention will be more fully understood from the following detailed description of applications thereof, taken together with the drawings, in which:

### BRIEF DESCRIPTION OF THE DRAWINGS

FIG. 1 is a schematic illustration showing a system for use with a subject who has overactive bladder (OAB), in accordance with some applications of the present invention; and

FIG. 2 shows a method for assessing treatment of a subject who has OAB, in accordance with some applications of the present invention.

### DETAILED DESCRIPTION OF EMBODIMENTS

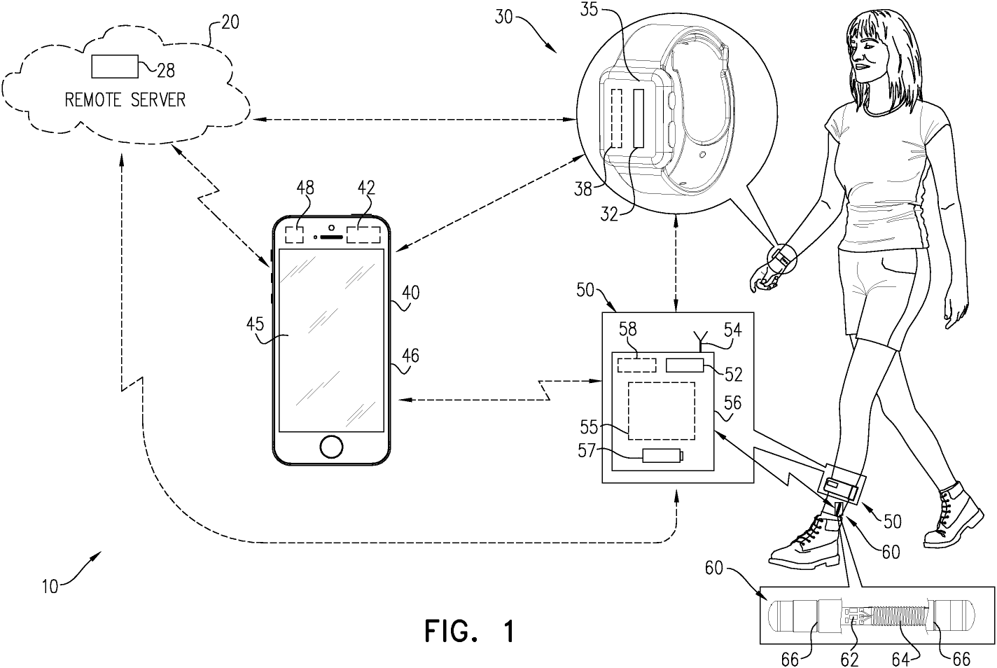

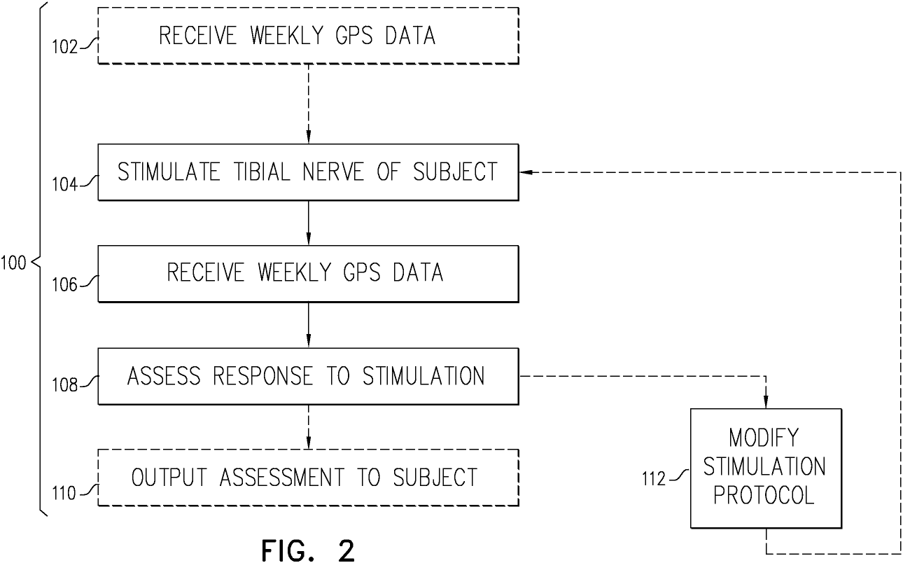

Reference is made to FIG. 1, which is a schematic illustration showing a system 10 for use with a subject who has overactive bladder (OAB), and to FIG. 2, which shows a method 100 for use with the subject, in accordance with some applications of the invention.

As shown, system 10 comprises a plurality of components, which are typically in communication (e.g., wireless communication) with each other, as indicated by the dotted arrows in FIG. 1. As described hereinbelow with reference to FIG. 2, system 10 is used to assess a response of the subject to a treatment for OAB, in response to data collected by one or more sensors that indicate activity of the subject over a time period extending over at least one week.

For some applications, the one or more sensors comprise at least one sensor that does not comprise a toilet-use monitor or other means for directly indicating a frequency of the subject’s voiding episodes. In a particular application, none of the sensors comprises a toilet-use monitor or other means for directly indicating a frequency of the subject’s voiding episodes. Typically for such applications, the sensors collect data that reflect an effect of the treatment upon the subject’s activity (e.g., the subject’s motion). It is hypothesized by the inventors that the data related to the subject’s activity reflect aspects of the subject’s response to treatment for OAB that are complementary to those aspects reflected by the subject’s voiding behavior.

System 10 comprises an implant 60 that delivers the treatment. Implant 60 is typically implanted adjacent to a tibial nerve of the subject, in order to stimulate the tibial nerve. As shown, the implant comprises at least one implant antenna 64 that receives wireless power, and implant circuitry 62 that uses the received wireless power to drive one or more electrodes 66 to apply a current to the tibial nerve.

For some applications, implant 60 is similar to one or more of the implants described in U.S. Pat. 9,713,707 to Oron et al. (e.g., implants 40, 60, 80, 100, 120, 140, 160, 180, 200, 220 described therein), which is incorporated herein by reference.

System 10 further comprises a portable controller 50 that has a battery 57 that stores battery power, and controller circuitry 52 that uses the battery power to drive a controller antenna 54 to transmit wireless power to implant antenna 64.

In this way, portable controller 50 wirelessly powers implant 60 to stimulate the tibial nerve, typically according to a stimulation protocol. Typically, the stimulation protocol is predetermined (e.g., at the time of manufacture, or by the subject’s physician). For some applications, the stimulation protocol may be modified (step 112 of method 100), as described hereinbelow with reference to FIG. 2.

System 10 further comprises an electronic processor that receives data from the sensors. For some applications, an electronic processor 28 is located at a remote server 20 that is in communication with other components of system 10 (e.g., portable controller 50 and/or a relay unit 40 that relays the data from the sensors to the portable controller). Alternatively, an electronic processor 38 may be housed within a wearable electronic device 30 (e.g., a watch, as shown) that is in communication with one or more other components of system 10. Alternatively still, an electronic processor 48 may be housed within a housing 46 of relay unit 40, and/or an electronic processor 58 may be housed within a housing 56 of portable controller 50.

In response to the data that electronic processor 28, 38, 48, 58 receives from the sensors, the electronic processor assesses a characteristic of a response of the subject to the stimulation (step 108 of method 100). For example, a quality-of-life score may be calculated, based on the data, in order to indicate the subject’s level of recovery from OAB.

For some applications, an indication of the assessment of the subject’s response to the stimulation (e.g., the quality-of-life score and/or a recommendation to consult with a caregiver) is then outputted to the subject (step 110 of method 100), e.g., via a user interface 35, 45, 55, such as a touchscreen, respectively of wearable electronic device 30, relay unit 40 or portable controller 50.

For some applications, system 10 receives data from one or more sensors that are external to system 10 (e.g., off-the-shelf sensors that are purchased separately from system 10), and these sensors transmit the data (e.g., by wireless communication) to remote server 20, relay unit 40 and/or directly to portable controller 50.

For some applications and as shown, wearable electronic device 30 houses a sensor 32. Alternatively or additionally, relay unit 40 comprises a personal electronic device (e.g., a smartphone, as shown) that houses a sensor 42.

The sensors typically sense data that indicate the subject’s activity over a time period extending over at least one week. For some applications, the sensors may transmit the data continuously, in real time. For some applications, the sensors comprise a memory storage component, in order to store the data until the data is transmitted (e.g., transmitting data when the sensors are in wireless communication with a wireless network and/or with another component of system 10).

The data may indicate different aspects of the subject’s activity, and therefore different sensors may be used with system 10. For some applications, the data may indicate aspects of the subject’s motion. For example, the one or more sensors may comprise a GPS signal receiver, a gyroscope, and/or an accelerometer. For some applications, the data may indicate other aspects of the subject’s activity. For example, the one or more sensors may comprise a sleep monitor (such as the sleep logbook disclosed in U.S. Pat. 7,572,225 to Stahmann et al.), a toilet-use monitor (such as the toilet monitor disclosed in U.S. Pat. 10,385,559 to Canfield et al.) and/or a microphone.

FIG. 2 shows a method 100 for assessing treatment of a subject who has OAB. Implant 60 is used to stimulate the tibial nerve of the subject (step 104), and electronic processor 28, 38, 48, 58 receives from the sensors data (e.g., GPS data, as shown in step 106) that indicate the subject’s activity over at least one week.

Steps 104 and 106 are not necessarily performed in this order. That is, (i) stimulation may be provided simultaneously with receiving data, (ii) the data may be received prior to the first time that the tibial nerve is stimulated using implant 60 (e.g., prior to implantation of the implant), and/or (iii) the data may be received after stimulating the tibial nerve. For some applications, data received prior to stimulating the tibial nerve serve as a baseline for comparison with data received after stimulating the tibial nerve, as described hereinbelow with reference to step 108.

As described below, for some applications, the subject’s response to the stimulation is assessed independently of direct indications of the subject’s voiding behavior. Typically for such applications, the received data do not directly indicate a frequency of the subject’s specific voiding episodes.

Alternatively or additionally, the data may directly indicate the frequency of the subject’s specific voiding episodes (e.g., via monitoring toilet flushes or nocturnal wakefulness episodes (e.g., bed-exits)). It will be appreciated that some particular toilet flushes are not indicative of OAB (e.g., the toilet may be flushed after cleaning of the toilet, and not because of voiding), and some bed-exits are not due to OAB (e.g., the subject may have left her bed during the night to check on a family member). Nevertheless, for the purposes of the present application, it is generally assumed that toilet flushes and nighttime bed-exits are particularly useful direct indicators of the state of a subject’s OAB.

Typically and as mentioned hereinabove, at least some of the received data indicate other aspects of the subject’s activity. It is hypothesized by the inventors that the subject’s activity level correlates positively with the subject’s response to OAB treatment. Therefore, for some applications, the received data indicate aspects of the subject’s motion. For some such applications, the received data (e.g., accelerometer-derived data) indicate a number of steps taken by the subject or a cumulative duration of a plurality of periods of the subject’s motion. For some such applications, the received data (e.g., GPS-derived data) indicate a distance traveled by the subject (e.g., by vehicular transport or by foot).

It is further hypothesized by the inventors that time spent by the subject in locations where bathrooms may not be immediately accessible correlates with a positive response to OAB treatment. Therefore, the received data may indicate a cumulative duration of time during which the subject is not (a) at home (b) in the subject’s workplace, or (c) indoors.

Typically, the electronic processor is used to assess the subject’s response to the stimulation, based upon the received data (step 108).

For some applications, the subject’s response to the stimulation is assessed by comparing the received data to (i) a baseline value and/or (ii) a predetermined threshold value.

For some such applications in which the data indicate a distance traveled by the subject, the subject’s response to the treatment is assessed by comparing a post-treatment-initiation distance traveled by the subject subsequently to the first time that the tibial nerve is stimulated using the implant to (i) a baseline distance traveled by the subject prior to the first time that the tibial nerve is stimulated using the implant, and/or (ii) a predetermined threshold distance.

For some such applications, a positive result of the stimulation is identified at least in part by the post-treatment-initiation distance being greater (e.g., at least 50 percent greater) than the threshold distance during a given time period. For example, for applications in which the distance is a distance travelled by vehicular transport, the threshold distance may be at least 50 km. For example, for applications in which the distance is a distance travelled by foot, the threshold distance may be at least 1 km.

For some applications, the stimulation protocol is altered in response to the subject’s response to the treatment, in order to optimize the treatment over time. That is, the stimulation protocol (e.g., a first stimulation protocol) may be modified (optional step 112) in response to the assessment of the subject’s response to the first stimulation protocol during a first time period, resulting in a second stimulation protocol. For example, the second stimulation protocol may call for a change in a number of daily or weekly stimulation sessions (e.g., addition of an evening stimulation session to a morning stimulation session). Alternatively or additionally, the second stimulation protocol may include a change in the internal operation of implant 60, such as, for example, an amplitude of individual current pulses, or a pulse frequency of the applied current. For some applications, the second stimulation protocol is outputted to the subject (e.g., via user interfaces 35, 45, 55, respectively of wearable electronic device 30, relay unit 40 or portable controller 50).

Typically for applications in which a second stimulation protocol is generated, implant 60 is then used to stimulate the tibial nerve (step 104) during a second time period, according to the second stimulation protocol. Further typically, the sensors then transmit data indicating activity of the subject (step 106) over the second time period to electronic processor 28, 38, 48, 58, based upon which the electronic processor assesses the response of the subject to the stimulation (step 108) over the second time period (e.g., as described hereinabove with reference to the first time period).

As described hereinabove, for some applications, electronic processor 28, 38, 48, 58 then outputs the assessment to the subject (step 110). For some such applications, electronic processor 28, 38, 48, 58 may output to the subject a recommendation to consult with a caregiver, responsively to the assessment (for example, such that a parameter of the treatment may be changed, e.g., addition of a stimulation session, as described hereinabove and/or a change in the internal operation of implant 60).

For some applications, the subject’s response to stimulation during the second time period may be compared to the subject’s response to stimulation during the first time period. For example, the data received during the first time period may be compared to data received during the second time period. Alternatively or in addition, the electronic processor may compare a first parameter (e.g., a first quality-of-life score) characterizing the subject’s response to stimulation during the first time period to a second parameter (e.g., a second quality-of-life score) characterizing the subject’s response to stimulation during the second time period. For some such applications, the comparison is then outputted to the subject (step 110) and/or the stimulation protocol may be further modified (step 112).

Applications of the invention described herein can take the form of a computer program product accessible from a computer-usable or computer-readable medium (e.g., a non-transitory computer-readable medium) providing program code for use by or in connection with a computer or any instruction execution system, such as electronic processor 28, 38, 48, 58. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., electronic processor 28, 38, 48, 58) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the methods described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., electronic processor 28, 38, 48, 58) or other programmable data processing apparatus, create means for implementing the functions/acts specified in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the present patent application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the present application.

Electronic processor 28, 38, 48, 58 and the other electronic processors described herein are typically hardware devices programmed with computer program instructions to produce a special purpose computer. For example, when programmed to perform the algorithms described herein, the electronic processor typically acts as a special purpose electronic processor. Typically, the operations described herein that are performed by electronic processors transform the physical state of a memory, which is a real physical article, to have a different magnetic polarity, electrical charge, or the like depending on the technology of the memory that is used.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for assessing treatment of a subject who has overactive bladder (OAB), the method comprising:
using an implant, stimulating a tibial nerve of the subject according to a stimulation protocol; and
using an electronic processor:
receiving data from one or more sensors, the data being indicative of activity of the subject over a time period extending over at least one week, and
based on the data, assessing a characteristic of a response of the subject to the stimulation.

2. The method according to claim 1, wherein receiving the data comprises receiving data not indicative of a frequency of specific voiding episodes of the subject.

3. (canceled)

4. The method according to claim 1, wherein receiving the data comprises receiving data indicative of a cumulative duration of time during which the subject is not at home.

5. The method according to claim 1, wherein receiving the data comprises receiving data indicative of a cumulative duration of time during which the subject is not in a workplace of the subject.

6. The method according to claim 1, wherein receiving the data comprises receiving data indicative of a cumulative duration of time during which the subject is not indoors.

7. The method according to claim 1, wherein receiving the data comprises receiving data indicative of a cumulative duration of a plurality of periods of motion of the subject.

8-9. (canceled)

10. The method according to claim 1, further comprising outputting to the subject an indication of the assessment.

11. The method according to claim 1, further comprising, responsively to the assessment, outputting to the subject a modification of the stimulation protocol.

12. (canceled)

13. The method according to claim 1, wherein receiving the data comprises receiving data indicative of a level of physical activity of the subject.

14-16. (canceled)

17. The method according to claim 13, wherein receiving the data comprises receiving smartphone-derived data.

18-22. (canceled)

23. The method according to claim 1, wherein receiving the data comprises receiving the data from the one or more sensors:
prior to a first time that the tibial nerve is stimulated using the implant, and
subsequently to the first time that the tibial nerve is stimulated using the implant.

24-27. (canceled)

28. The method according to claim 1, wherein receiving data from the one or more sensors comprises receiving data indicative of a distance traveled by the subject.

29-30. (canceled)

31. The method according to claim 28, wherein receiving the data indicative of the distance traveled comprises receiving GPS-derived data.

32. The method according to claim 28, wherein assessing the characteristic of the response of the subject to the stimulation based on the data comprises identifying a positive result of the stimulation based at least in part on a post-treatment-initiation distance traveled by the subject subsequently to the first time that the tibial nerve is stimulated using the implant being greater than a predetermined threshold distance during a given time period.

33-38. (canceled)

39. The method according to claim 1, further comprising, based on the data, calculating a quality-of-life score indicative of a level of recovery of the subject from OAB.

40-42. (canceled)

43. The method according to claim 1, wherein:
the time period is a first time period,
the stimulation protocol is a first stimulation protocol,
the step of assessing comprises assessing the characteristic of the response of the subject to the stimulation over the first time period, and the method further comprises:
responsively to the step of assessing, determining a second stimulation protocol,
using the implant, stimulating the tibial nerve according to the second stimulation protocol over a second time period extending over at least one week,
using the electronic processor, receiving a second set of data from the one or more sensors, the data being indicative of activity of the subject over the second time period, and
based on the second set of data, assessing the characteristic of the response of the subject to the stimulation over the second time period.

44. The method according to claim 43, wherein:
stimulating the tibial nerve according to the first stimulation protocol comprises stimulating the tibial nerve during a first number of daily stimulation sessions, and
the step of stimulating the tibial nerve according to the second stimulation protocol comprises stimulating the tibial nerve during a second number of daily stimulation sessions.

45. (canceled)

46. The method according to claim 43, wherein determining the second stimulation protocol comprises determining a change in a parameter of internal operation of the implant.

47. The method according to claim 46, wherein:
stimulating the tibial nerve according to the first stimulation protocol comprises stimulating the tibial nerve at a first amplitude of individual current pulses,
determining the change in the parameter comprises determining a change in the amplitude of individual current pulses, and
the step of stimulating the tibial nerve according to the second stimulation protocol comprises stimulating the tibial nerve at a second amplitude of individual current pulses.

48. The method according to claim 46, wherein:
stimulating the tibial nerve according to the first stimulation protocol comprises stimulating the tibial nerve at a first pulse frequency of applied current,
determining the change in the parameter comprises determining a change in the pulse frequency of applied current, and
the step of stimulating the tibial nerve according to the second stimulation protocol comprises stimulating the tibial nerve at a second pulse frequency of applied current.

49-67. (canceled)
